(19) United States
(12) Patent Application Publication
(10) Pub. No.: US 2021/0295466 A1
(43) Pub. Date: Sep. 23, 2021

(54) SHAPE CONFORMING PROJECTIONS OF MEDICAL INFORMATION

(71) Applicant: Summit Technology Laboratory, Irvine, CA (US)

(72) Inventors: Lohrasb Ross Sayadi, Irvine, CA (US); Mahdi Abbaspour Tehrani, Irvine, CA (US); Alexander Sidenko, Irvine, CA (US); Muhammad Twaha Ibrahim, Irvine, CA (US)

(21) Appl. No.: 17/337,852

(22) Filed: Jun. 3, 2021

Related U.S. Application Data
(63) Continuation-in-part of application No. 16/584,481, filed on Sep. 26, 2019.
(60) Provisional application No. 62/737,680, filed on Sep. 27, 2018.

(51) Int. Cl. G06T 3/00; G06T 19/20; A61B 90/00

(52) U.S. Cl. CPC G06T 3/005; G06T 19/20; A61B 90/36; A61B 2090/368; G06T 2219/2004; A61B 2090/366; A61B 2090/395; G06T 2210/41

(57) ABSTRACT

A projection system for projecting medical information onto the surface of the human body such that the medical information conforms and registers to the contour of the underlying shape of the region it is being projected upon. The projection system is in communication with one or more projectors and one or more sensors which capture the topography/area/volume of the region of interest and/or the view of the user, and alter the medical information such that the medical information remains accurate in dimensions when projected into the area of interest.

### CROSS-REFERENCES TO RELATED APPLICATIONS

This application is a continuation-in-part and claims benefit of Non-Provisional U.S. patent application Ser. No. 16/584,481 filed Sep. 26, 2019 which claims benefit of Provisional U.S. Patent Application No. 62/737,680 filed Sep. 27, 2018, the specification(s) of which is/are incorporated herein in their entirety by reference.

### BACKGROUND OF THE INVENTION

### Field of the Invention

The present invention relates to projection of medical information onto the human body in such a way that the projected information conforms to the shape of the area being projected upon.

### Background Art

In the medical field physicians rely on a whole host of medical information in order to make decisions. For example, surgeons can refer to different images (CT scans, X-rays, MRI's, IR maps etc.) and computer-generated operative plans to help guide decision making in the operating room. However, in its current state, medical information can only be displayed for physicians on either paper, computer screen or through a mixed reality head mounted device such as Holo-Lens. The very primitive paper or computer displays which offer visualization of medical information, have a significant limitation in that the operator must mentally manipulate and estimate what he/she sees on the screen to the actual patient. Similarly, in sterile environments such as the operating theatre, paper and computer displays cannot reside in local operative fields making the medical information less accessible.

On the other hand, mixed reality technology which uses a head mounted device, displays the information to the physician virtually into the operative field, solving the issue of accessibility. However, these are personalized systems where the experience is only for the wearer of the head mounted display and have inherent problems such as: alignment/registration issues, the need to wear the technology on the head which may cause user discomfort, limited interaction with the virtual image, and display of information only to the user with the wearable device. Given the issues at hand, a new system is needed to display medical information for medical professionals which does not rely on wearable devices and allows for accurate registration and conformation of medical information onto the patient anatomy.

Prior systems for image projection of anatomical features on a subject, such as U.S. Publication No. 2017/0165028 A1 (hereinafter “Hummelink”), comprise a plurality of intraoperative position markers, a moveable first detector, a control unit arranged for receiving one or more preoperative images comprising one or more anatomical features of interest, and a moveable projector, wherein the system is capable of projecting a 3D image to fit onto a body surface by taking into account only positioning and orientation of the original image. For example, paragraph [0047] of Hummelink recites that “a correct position and orientation of a projected image can . . . be achieved when the projection system 1 comprises at least three intraoperative position markers 10 (using triangulation detection methods).” However, systems that only take positioning and orientation of the original image into account are unable to maintain ALL dimensions of the original information after projection (i.e. length, width, angle, and positioning of the information) regardless of the region or anatomy being projected upon.

The present invention features a system that allows for medical information to be projected onto the human body while remaining accurate and precise without distortion due to non-flat shape of the underlying part of the human body, objects in the operative field, or movement. Furthermore, the user can interact with the projected information through hand gestures, laser pointers etc. to move and alter the information while maintaining contour conformation.

### BRIEF SUMMARY OF THE INVENTION

In some aspects, the present invention comprises of a projection system which is used to project medical information onto the surface of the human body such that the medical information conforms and registers to the contour of the underlying shape of the region it is being projected upon. The projection system is in communication with one or more projectors and one or more sensors (e.g. visible camera, depth camera, IR camera, head/eye tracker) which capture the topography/area/volume of the region of interest and/or the view of the user, and alter the medical information such that the medical information remains accurate in dimensions (length, width, angles, positioning etc.) when projected into the area of interest. The medical information also adapts to the movements of the underlying surface while maintaining shape conformation. This projected information can be altered in real time (moved, repositioned, stretched, lengthened, shortened) by the user while maintaining shape conformation using modalities such as but not limited to hand gestures, markers or digital pens, tablets or other mobile devices. The projected information can change based on the viewpoint of the user. Finally, the projected information can interact with a dye (surface agent) placed on the area being projected upon which marks (i.e. temporary tattoos) the information onto the surface being projected upon.

An inventive technical feature of the presently claimed invention is the ability to maintain dimensions of the original information after projecting the said information onto a physical surface. Without wishing to limit the invention to any theory or mechanism, it is believed that the technical feature of the present invention advantageously provides for the present invention to project information onto a 3D surface such that it appears on the 3D surface in the same way that it would appear on a 2D computer display. For example, if the image has two intersecting lines, the projection of these lines will become curves when projected on a curved 3D surface. The length/angle of lines in the image will become the length of the curves and the angle of intersection of the curves in the projection. None of the presently known prior references or work has the unique inventive technical feature of the present invention.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIGS. 1A-1B show surgical marking (dotted line) for breast reconstruction being projected upon by the present invention. As seen in FIG. 1B, the dotted line surgical markings conform to the region being projected upon (breast) such that the surgical marking bends around the contour of the breast. Without such a system, significant distortion of the medical information may be encountered, altering the accuracy of the surgical markings.

FIG. 2 shows a flowchart of a method of generating and calibrating a digital model to be projected onto a physical model, wherein the digital model conforms to a shape of the physical model while maintaining accuracy of the original shape.

FIG. 3A shows a projection of a checkerboard pattern onto the breast without shape conformation. As seen in the image, the checkerboard pattern is distorted by the contour of the breast causing the angles of each box to be obtuse angles rather than 90 degrees.

FIG. 3B shows a projection of a checkerboard pattern onto the breast with shape conformation. As seen in the image, the checkerboard pattern conforms to the contour which is demonstrated by the ability to maintain the angles of each box at 90 degrees.

FIG. 4 shows an embodiment of the system comprising one or more tiled (to increase coverage or resolution) or superimposed (to increase brightness) projectors, and one or more sensors (e.g. visible camera, depth camera or IR cameras). It also shows the interactive hand or device (e.g. ruler, laser pointer, scalpel).

FIG. 5 shows a non-limiting embodiment of the system that includes a head/eye tracking device and a projection unit.

FIG. 6 shows another non-limiting embodiment of the system that includes multiple ceiling-mounted head/eye tracking devices and a projection unit.

### DETAILED DESCRIPTION OF THE INVENTION

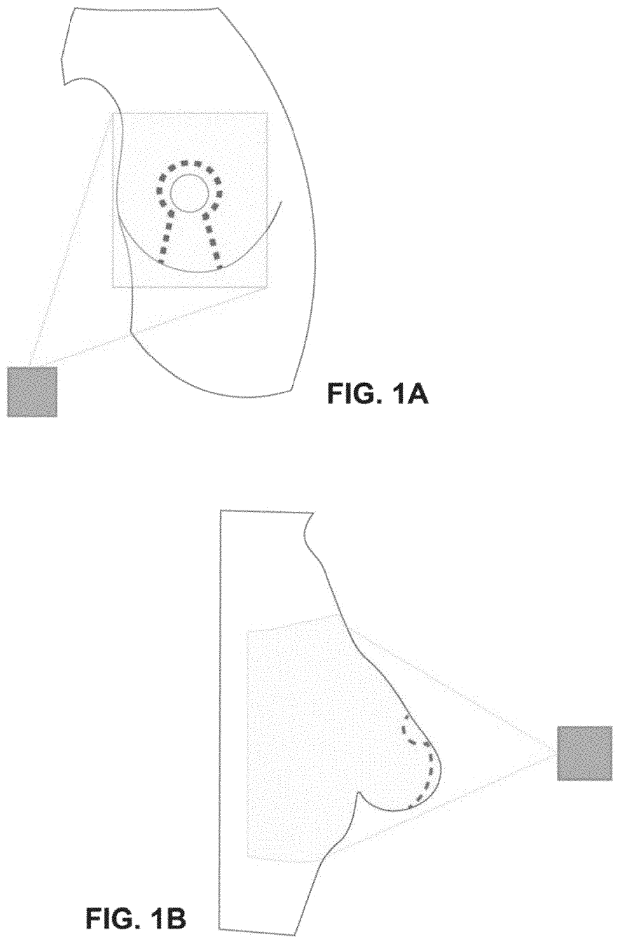

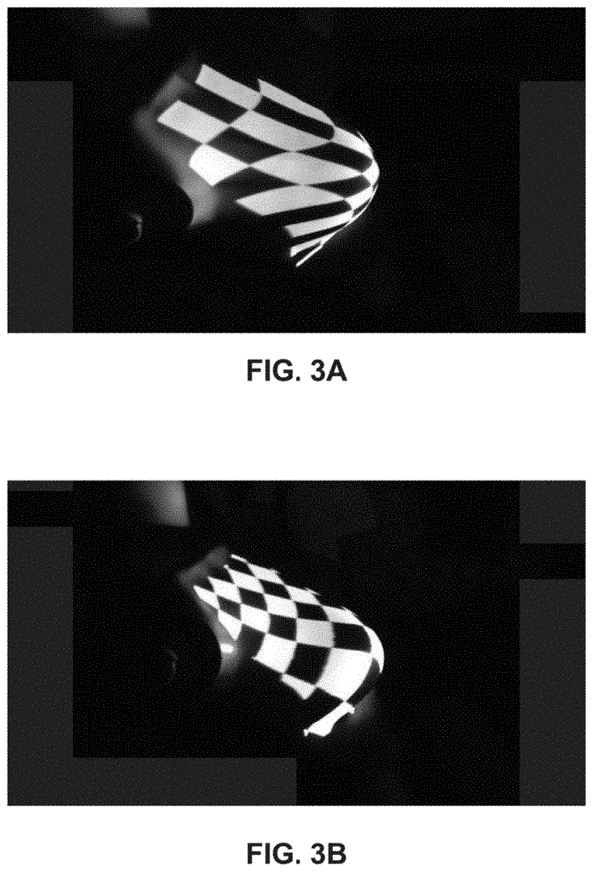

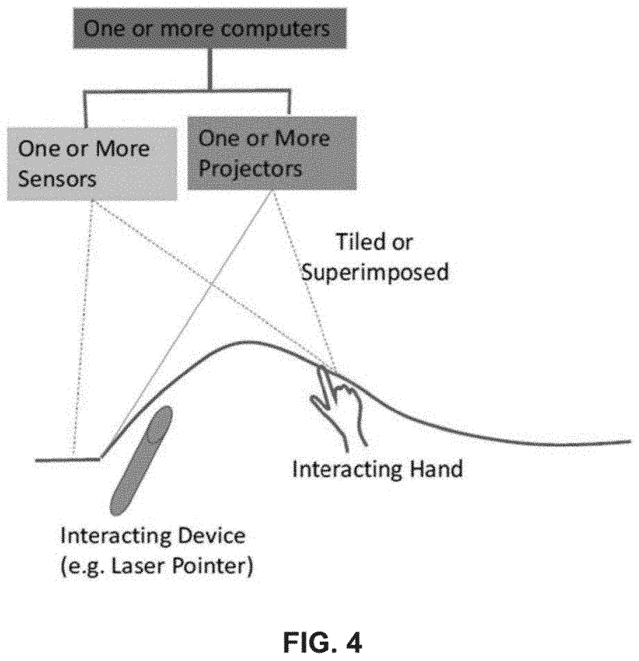

Referring now to FIGS. 1A-6, in some embodiments, the present invention features a system and method of projecting any medical information (i.e. CT scans, X-rays, blood flow map, thermal maps, surgical guides) onto the anatomy (or anatomical models) such that the projection both registers and conforms to the contour of the anatomical region it is being projected upon (i.e. nose, cheeks, breasts, chest, ears etc.), thereby maintaining the accuracy (length, angles positioning) of the medical information. In some embodiments, a length, width, angle, and positioning of the information may be maintained while projected, regardless of the region or anatomy being projected upon.

The various dimensions of the information being projected may be maintained through markings on the physical anatomy of the surface being projected on. The canonical template anatomy (e.g. face) has the medical information (e.g. surgical cut lines) whose geometric features have to be maintained when projected on the physical model. The alignment (position, orientation, size, etc.) of the canonical model to the 3D digital model of the physical model is computed. Since the 3D digital model is a continuous digital reconstruction of the physical model, the alignment between the 3D digital model and the physical model is also known. Since the physical model is continuously monitored by the camera, the relative position and orientation of the camera with respect to the physical model is computed (camera calibration). Finally, the projector and camera pair are also calibrated with respect to each other. Combining all the above inter-relationship between the medical information, models, and the devices—from the images on the canonical template to the projector—a correct image that needs to be projected to preserve the features of the medical information can be computed.

In another aspect of the present invention, medical information (i.e. CT scans, X-rays, blood flow map, thermal maps, surgical guides) is projected onto the anatomy (or anatomical models) such that the projection adapts to the movement of the surface it is being projected upon (breathing, facial expression) while maintaining conformation to the underlying shape. This adaptation may be carried out by detecting, by a motion sensor, any motion of the underlying shape. Additionally, the present invention may adapt to changes of the underlying shape on a fixed time interval.

In some embodiments, medical information (i.e. CT scans, X-rays, blood flow map, thermal maps, surgical guides) is projected onto the anatomy (or anatomical models) such that the projection can be altered by the user (i.e. stretched, scaled, rotated, repositioned) while maintaining conformation to the underlying shape. The medical information is changed either on the canonical template, or the 3D model, or on the canonical template after it has been morphed to align with the 3D model, or on the physical model. Since the inter-relationship between all these representations are known or computed, the (modified) medical information can be transferred from one representation to the other seamlessly using the transformations between these representations. Finally, the devices (cameras and projectors) are also calibrated with respect to the physical model. So, the images that need to be projected can also be computed the same way as the original medical information was projected.

In other embodiments, medical information (i.e. CT scans, X-rays, blood flow map, thermal maps, surgical guides) is projected onto the anatomy (or anatomical models) such that the projection is correct from the single preferred user's (i.e. surgeon) point of view and projection change with the user's head position, in order to correctly visualize internal data (e.g. bones, blood vessels, organs) on the surface.

In some other embodiments, medical information (i.e. CT scans, X-rays, blood flow map, thermal maps, surgical guides) is projected onto the anatomy (or anatomical models) such that the projected information can be adapted to best fit the shape of the area projected upon by integrating contour, shape and volume of the region being projected upon into mathematical consideration.

In additional embodiments of the present invention, projected medical information can be interacted with by the user via multiple interaction modalities like laser pointers, hand gestures, markers or digital pens, scalpels, tablets or other mobile devices while maintaining conformation to the underlying shape. These multiple interaction modalities may be used to transform the position or shape of the projection (i.e. rotating, repositioning, scaling, stretching) and/or change the color of the projection.

In further embodiments of the present invention projected medical information can interact with a surface agent, such as a light activated dye or heat activated dye, placed on the area being projected upon which marks (i.e. temporary tattoos) the information onto the surface being projected upon.

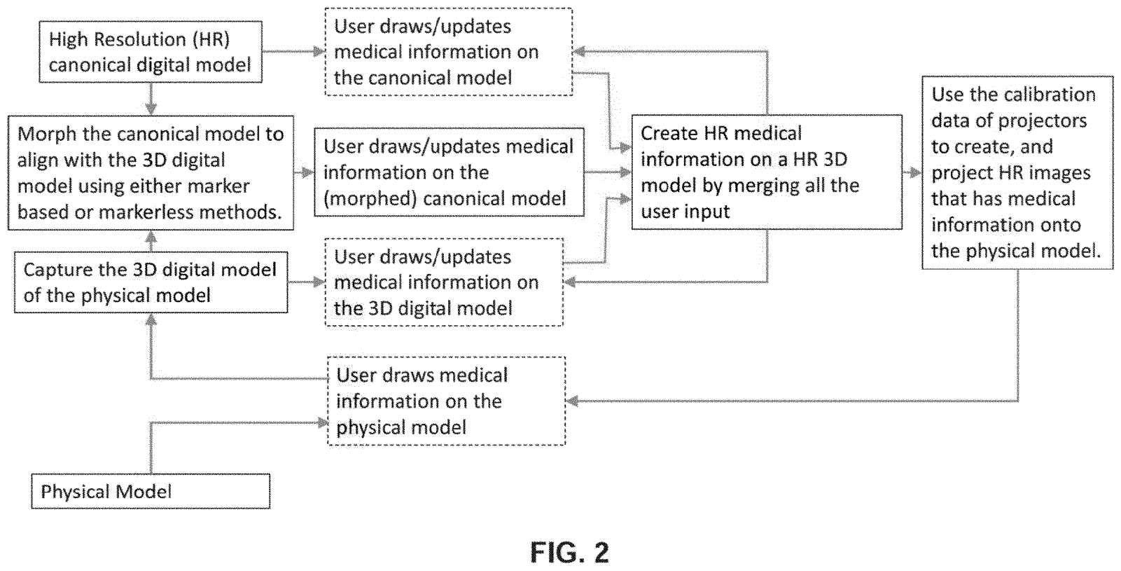

Referring now to FIG. 2, the present invention features a method of projecting medical information onto the body of a subject such that the information being projected conforms to a shape of the physical model while maintaining accuracy of the original shape. In some embodiment, the method may comprise generating or capturing a high resolution canonical model that represents the physical model, capturing a digital model of the physical model, and morphing the canonical model to align with the digital model to create a morphed canonical model. The method may further comprise drawing medical information onto the canonical model, the morphed canonical model, or the digital model or the physical model. The method may further comprise transferring the updated medical information to one or more representations including canonical, morphed canonical, and digital models. The method may further comprise projecting, by one or more projectors, one or more images of the morphed canonical model onto the physical model. The images may be created using a plurality of calibration data of the one or more projectors to create a faithful visualization of the medical information on the physical model. Faithful visualization may consist of maintaining a length, width, angle, and positioning of medical information on the physical model. In some embodiments, the method may further comprise comparing the medical information on the digital model with the medical information on the morphed canonical model.

In some embodiments, the method may further comprise repeating the steps of capturing a digital model of the physical model including the information projected on the physical model and morphing the canonical model to align with the digital model to create a morphed canonical model to refine the morphing of the canonical model or the camera and projector calibration or the digital model so that the projected images are more accurate with respect to the physical model and/or to project the updated medical information, and projecting, by one or more projectors, one or more images of the morphed canonical model onto the physical model. In some embodiments, morphing the canonical model to align with the digital model may be executed through the use of marker-based methods or markerless methods. In some embodiments, the projected digital model may adapt to a movement of a viewpoint of the user.

### Example

The following is a non-limiting example of the present invention. It is to be understood that said example is not intended to limit the present invention in any way. Equivalents or substitutes are within the scope of the present invention.

Example 1: A surgeon may acquire medical information such as blood flow and vascularity of a patient's breast tissue using and infrared camera or dye-based imaging system and project this medical information on the patient's breast tissue in such a way that the projection conforms to the contour of the breast such that the areas of poor blood flow are accurately demarcated using the projection system. The area of poor blood flow which is projected can be traced using a marker. The traced region can then be surgically removed by the surgeon.

Example 2: A surgeon may acquire information on the location of major blood vessels in the abdomen using CT scans prior to performing surgery. Intraoperatively the physician can project the preoperative CT scan onto the abdomen in such a way that the projection conforms to the exact contour of the patient's abdomen as well as the viewpoint of the surgeon. Using this technique, the physician can actually identify the location of the major blood vessels for surgical dissection.

Example 3: Referring to FIGS. 1A-1B, a surgeon may create a computerized patient specific stencil for breast surgery before surgery which they can use during surgery to make their surgical markings. The surgeon can project this stencil on the breast intraoperatively. The computerized template remains accurate (length, angles) while conforming to the area of the breast it is projected upon. The surgeon can alter the projected stencil (stretch, rotate, alter limbs of the stencil) using hand gesture, laser pointer etc. All the while the image shape conforms as it is being altered.

Example 4: A surgeon performing craniofacial surgery may project a virtual plan onto the surface of the skull guiding them where to make cuts into the mandible. The projected information registers and conforms to the contour of the mandible maintaining accuracy of the cutting locations that were planned before surgery.

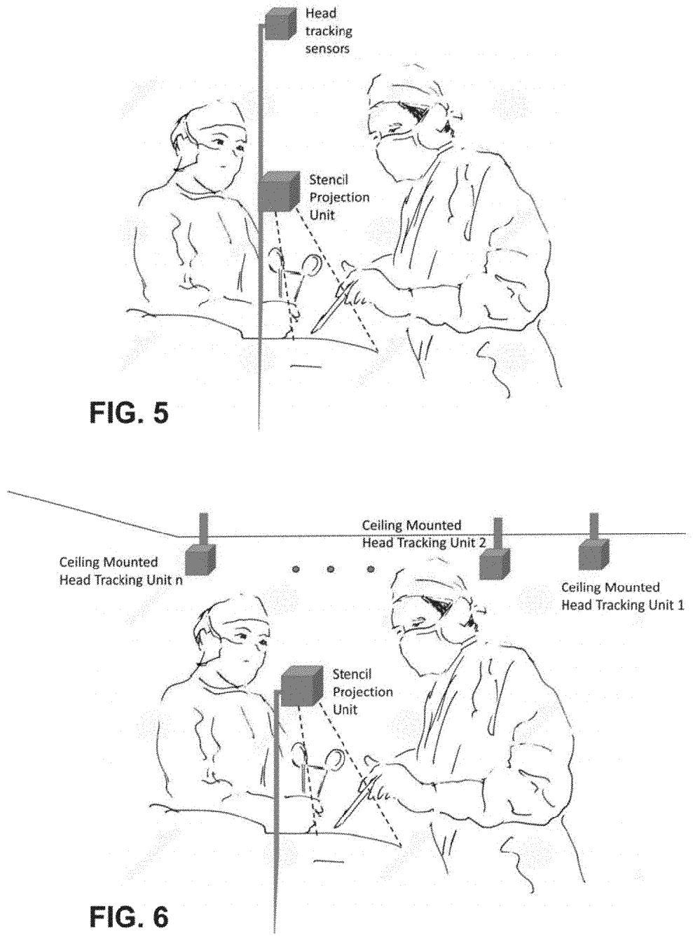

Example 5: Referring to FIGS. 5-6, a medical student can project an X-ray of the chest onto the chest of an anatomical model such that the projected X-ray registers and conforms to the shape of the chest of the anatomical model and from the student's perspective.

Example 6: Projection onto a physical model could be used to execute a virtual surgery simulation. For example, information may be projected onto an uncut physical body, and the projected information may interact with interaction modalities, such as a scalpel, to simulate cuts and the resulting organs that would be shown from the cut without actually requiring the body to be cut. The effects of the cuts are simulated and the graphics rendering of the internal features after the virtual cut are additionally simulated.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. A method of projecting medical information onto body of a subject such that the information being projected conforms to a shape of a region or anatomy being projected upon while maintaining accuracy of the original shape, wherein:
a. projected information registers or aligns to the region/anatomy being projected upon, wherein a length, width, angle, and positioning of the information is maintained while projected, regardless of the region or anatomy being projected upon;
b. projected information is configured to adapt to movement of the projection surface;
c. projected information is altered by the user while maintaining conformation to the underlying shape;
d. projected information is adapted to best fit the shape of the region/anatomy projected upon by integrating contour, shape or volume of the region being projected upon into mathematical consideration; and
e. projected information is configured to be interacted with by the user via multiple interaction modalities.

2. The method of claim 1, wherein the projected image interacts with a surface agent placed on the area being projected upon which marks the information onto the surface being projected upon.

3. The method of claim 1, wherein the projected information adapts to a movement of a viewpoint of the user.

4. A method of projecting medical information onto body of a subject such that the information being projected conforms to a shape of the physical model while maintaining accuracy of the original shape, the method comprising:
a. generating or capturing a high resolution canonical model that represents the physical model;
b. capturing a digital model of the physical model;
c. morphing the canonical model to align with the digital model to create a morphed canonical model;
d. drawing medical information onto the canonical model, or the morphed canonical model, or the digital model, or the physical model;
e. transferring the updated medical information to one or more representations including canonical, morphed canonical, and digital models;
f. projecting, by one or more projectors, one or more images of the morphed canonical model onto the physical model, wherein the images are created using a plurality of calibration data of the one or more projectors to create a faithful visualization of the medical information on the physical model, wherein faithful visualization consists of maintaining a length, width, angle, and positioning of medical information on the physical model.

5. The method of claim 4 further comprises repeating steps b and c, and comparing the medical information on the digital model with the medical information on the morphed canonical model to refine the morphing of the canonical model or the camera and projector calibration or the digital model so that the projected images are more accurate with respect to the physical model.

6. The method of claim 4 further comprising repeating steps b-f to project the updated medical information.

7. The method of claim 4, wherein morphing the canonical model to align with the digital model is executed through the use of marker-based methods or markerless methods.

8. The method of claim 4, wherein the projected digital model adapts to a movement of a viewpoint of the user.
